(19) United States
(12) Patent Application Publication
(10) Pub. No.: US 2012/0178810 A1
(43) Pub. Date: Jul. 12, 2012

(54) Extended release formulation of an antiepileptic agent

(76) Inventors: Tailor Prakash Boya, Hyderabad (IN); Haranatha Babu Balanagu, Hyderabad (IN); Shailesh Suresh Bhamare, Hyderabad (IN); Kishor Dattatray Deo, Hyderabad (IN); Sivakumaran Meenakshisunderam, Hyderabad (IN)

(21) Appl. No.: 12/310,864

(22) PCT Filed: Sep. 10, 2007
(86) PCT No.: PCT/IB2007/002963

§ 371 Date: Nov. 7, 2009

(30) Foreign Application Priority Data
Sep. 11, 2006 (IN) 1644/CHE/2006

(51) Int. Cl. A61K 31/19; A61P 25/08; A61P 25/18; A61P 25/06

(52) U.S. Cl. 514/557

(57) ABSTRACT

The present invention relates to an extended release formulation of an antiepileptic agent. More particularly, the present invention relates to an extended release formulation of divalproex sodium. The present invention also relates to a process for the preparation of extended release formulation of divalproex sodium.

### FIELD OF THE INVENTION

The present invention relates to an extended release formulation of an antiepileptic agent. More particularly, the present invention relates to an extended release formulation of divalproex sodium.

The present invention also relates to a process for the preparation of extended release formulation of divalproex sodium.

### BACKGROUND OF THE INVENTION

Divalproex sodium disclosed in U.S. Pat. No. 4,988,731 and U.S. Pat. No. 5,212,326 is a stable co-ordination compound comprised of sodium valproate and valproic acid in a 1:1 molar relationship and formed during the partial neutralization of valproic acid with 0.5 equivalent of sodium hydroxide. Divalproex sodium is marketed under the trade name DEPAKOTE and DEPAKOTE ER® (Abbott) as delayed release and extended release tablets used for the treatment of epileptic seizures or convulsions, mania and migraine. Commercially available DEPAKOTE ER® tablets contain Divalproex sodium as active ingredient and excipients such as hydroxypropylmethylcellulose, lactose, microcrystalline cellulose, polyethylene glycol, potassium sorbate, propylene glycol, silicon dioxide, titanium dioxide, triacetin and FD & C Blue No. 1. In addition, 500 mg tablets contain iron oxide and polydextrose.

Valproic acid and its derivatives have a relatively short elimination half-life. Frequent dosing is thus necessary to maintain reasonably stable plasma concentrations. This leads to substantial fluctuations in the plasma concentration of the drug, especially in chronic administration which requires frequent dosing. This frequent dosing results in poor patient compliance. In view of these problems, extended release formulations have been developed to decrease the dosing frequency of such compounds.

The most common method for controlled release is to include the drug in a matrix system. Hydrophilic polymer matrix systems are widely used in oral controlled drug delivery because of their flexibility to obtain a desirable drug release profile, cost-effectiveness, and broad regulatory acceptance. The release of the drug from the hydrophilic matrix is by dissolution of drug from the core and its diffusion through the pores of the matrix.

Given below are the patents/patent publications, which disclose controlled release compositions of valproic acid and its salts:

U.S. Pat. No. 4,913,906 discloses a controlled release dosage form comprising an essentially homogenous admixture of valproic acid and its pharmaceutically acceptable salts and esters and biodegradable or non-biodegradable polymers.

U.S. Pat. No. 5,009,897 discloses the coated granules composition for compressing into tablets comprising core of divalproex sodium and coating comprising polymer selected from povidone, hydroxypropylcellulose and hydroxypropylmethyl cellulose and microcrystalline cellulose, wherein the polymer: microcrystalline cellulose ratio being from about 1:15 to about 2:1 by weight.

U.S. Pat. No. 5,019,398 discloses a sustained release tablet composition comprising divalproex sodium in a matrix of hydroxypropylmethylcellulose of viscosity from 8000 to 12000 mPa.s, and 8% to 10% by weight relative to the weight of the complex of hydrated silica.

U.S. Pat. No. 5,169,642 discloses a sustained release dosage form comprising granules of divalproex sodium or amides or esters of valproic acid coated with a sustained release composition comprising ethyl cellulose or a methacrylic methyl ester, a plasticizer, a detackifying agent, and a slow-release polymeric viscosity agent.

U.S. Pat. No. 6,419,953 and U.S. Pat. No. 6,511,678 discloses a controlled release dosage form comprising a hydrophilic matrix formed from a uniform admixture of divalproex sodium, hydrophilic polymer selected from the group consisting of polyvinylpyrolidine, hydroxypropyl cellulose, hydroxypropylmethylcellulose, methyl cellulose, vinyl acetate copolymers, polyethylene oxide, methacrylic acid copolymers, and maleic anhydride/methyl vinyl ether copolymers; and lactose, microcrystalline cellulose and silicon dioxde.

U.S. Pat. No. 6,150,410 discloses a composition comprising divalproex sodium dispersed or dissolved in polymer matrix comprising from about 10 weight percent to about 40 weight percent of a pharmaceutically acceptable neutral, water-swellable, hydrophilic polymer, and from about 20 weight percent to about 50 weight percent of a pharmaceutically acceptable acid soluble polymer which is water swellable above about pH 5, wherein all percentages are based upon the total weight of the formulation. This patent further discloses that at low pH values in the stomach, the acid soluble polymer is believed to have the greatest effect on release of the low soluble drug. In this environment, protonation of the acid-soluble polymer by the acidic gastric juices raises the pH in the local environment of the formulation matrix to levels where the acidic pharmacologic agent is more soluble thereby accelerating the release of drug at this pH.

US 2004/0037880 discloses an extended release pharmaceutical composition comprising: from about 10-90% of divalproex sodium, from about 7-65% of hydroxypropyl methylcellulose, from about 0.5-18% of lactose, and from about 0.5-5% colloidal silicon dioxide; wherein all percentages are based upon the total weight of the pharmaceutical composition; wherein the pharmaceutical composition is manufactured under controlled atmospheric conditions.

US 2005/0276849 discloses a sustained release oral dosage form comprising neutralized divalproex sodium; a solubility modulating agent; and a pharmaceutically acceptable carrier; said sustained release oral dosage form providing a Tmax at from about 4 to about 20 hours after oral administration of the dosage form to a human.

US 2007/0048379 discloses the process for preparing a pharmaceutical tablet comprising: (a) mixing a valproate compound with at least one excipient to form a premix; (b) adding a sufficient amount of water to the premix of step (a) and mixing to form a wet granulation; (c) drying the wet granulation of step (b) at a temperature and time sufficient to form granules, wherein the granules are essentially free of an organic solvent; (d) coating the granules of step (c) to form coated granules; and (e) compressing the coated granules of step (d) to form a tablet.

US 2007/0160667 discloses a controlled release dosage formulation comprising, a) a valproic acid compound in an amount of about 40% to about 80% by weight of the dosage form, and b) at least two polymers each in an amount of less than about 20% of the tablet weight and further comprising a granulate matrix surface coating.

WO 2004/078164 discloses a sustained release composition comprising core comprising drug, hydrophobic material excluding a polymer capable of swelling that causes disintegration of the tablet and water soluble low molecular weight excipient.

WO 2005/079753 discloses an extended release composition comprising divalproex sodium, high and low viscosity grade hydroxypropyl methylcellulose.

WO 2006/010995 discloses a controlled release composition comprising from about 20-80% w/w of divalproex sodium not more than 20% w/w of a pharmaceutically acceptable polymer and 10-50% w/w of filler; all weight percentages are based upon the total weight of the tablet dosage form.

WO 2006/025029 discloses an extended release pharmaceutical composition comprising divalproex sodium, hydroxypropyl methylcellulose, lactose and colloidal silicon dioxide wherein the amount of lactose is less than 8% w/w of the total weight of the pharmaceutical composition.

WO 2006/064321 discloses an oral controlled release composition from about 10-90% w/w of divalproex sodium, no more than 20% w/w of a pharmaceutically acceptable polymer, about 0.1 to 10% of a lubricant, and 2-25% w/w of filler; all weight percentages are based upon the total weight of the tablet dosage form.

The above prior art references discloses various controlled release formulations of divalproex sodium and other valproate compounds using hydrophilic polymers and combination with other excipients, that permits once-a-day dosing. The inventors of the present invention during their efforts to develop extended release formulation found that the presence of both hydrophilic and hydrophobic polymer in the composition effectively control the release of the drug from the dosage form over an extended period of time.

### OBJECTIVE OF THE INVENTION

Accordingly, the main objective of the present invention is to provide an extended release formulation of divalproex sodium.

Yet another objective of the present invention is to provide an extended release formulation of divalproex sodium in such a way that it will comply with the reference product in terms of in vivo parameters like Cₘₐₓ, tₘₐₓ and AUC and in vitro parameters like dissolution etc.

Yet another objective of the present invention is to provide process for the preparation of an extended release formulation of divalproex sodium.

### SUMMARY OF THE INVENTION

Accordingly, the main embodiment of the present invention is to provide an extended release formulation comprising divalproex sodium dispersed in a polymeric matrix comprising of about 5 to about 20% w/w of hydrophilic polymer, about 2 to about 20% w/w of hydrophobic polymer and about 15 to about 30% w/w of diluent.

### DETAILED DESCRIPTION OF THE INVENTION

In another embodiment, the present invention also provides an extended release formulation comprising divalproex sodium dispersed in a polymeric matrix comprising of about 5 to about 20% w/w of hydrophilic polymer, about 2 to about 20% w/w of hydrophobic polymer and about 15 to about 30% w/w of diluent, which exhibits the following dissolution profile when measured in a type 2 dissolution apparatus (paddle) at 100 rpm, at a temperature of 37±0.5° C., in 500 ml of 0.1 N HCl for 45 minutes, followed by 900 ml of 0.05 M phosphate buffer containing 75 mM sodium laurel sulfate (pH5.5):

i. not more than about 30% of total valproate is released after 3 hours of measurement;

ii. from about 30 to about 70% of total valproate is released after 9 hours of measurement;

iii. from about 50 to about 90% of total valproate is released after 12 hour of measurement, and;

iv. not less than 85% of total valproate is released after 18 hours of measurement.

In another embodiment of the present invention, the extended release formulation further comprises one or more excipients selected from binder, glidant and lubricant.

In another embodiment, the extended release formulation comprises at least 40% to about 70% w/w, preferably 50% to about 60% w/w of divalproex sodium.

Suitable hydrophilic polymers of the present invention include polyvinylpyrrolidone, hydroxypropylcellulose, hydroxypropylmethylcellulose, methylcellulose, hydroxyethylcellulose, vinyl acetate copolymers, alginic acid and its salts, xanthan gum and polyethylene oxide or combination thereof. The amount of hydrophilic polymer may range from about 5 to about 20% w/w, preferably about 10 to about 15% w/w.

Suitable hydrophobic polymers of the present invention include ethyl cellulose, cellulose acetate, polymethacrylic acid esters copolymer such as Eudragit NE3OD and Eudragit NE40D, copolymers of polyvinyl alcohol and high molecular weight polyvinylalcohols, glyceryl behenate, glyceryl palmitostearate, glyceryl monostearate and glyceryl distearate or combination thereof. The amount of hydrophobic polymer may range from about 2 to about 20% w/w, preferably about 5 to about 15% w/w.

The incorporation of hydrophobic polymer in addition to hydrophilic polymer controls the drug release to some extent, which could be attributed to the decreased penetration of the solvent molecules in the presence of hydrophobic polymer leading to decreased diffusion of the drug from the matrix thereby controlling the release of the drug from the dosage form over an extended period of time.

Suitable diluents of the present invention include calcium phosphate, calcium sulfate, microcrystalline cellulose, lactose, mannitol, pregelatinised starch, sorbitol and the like or combination thereof.

Suitable binders of the present invention include polyvinylpyrrolidone, xanthan gum, carboxymethylcellulose, methylcellulose, ethylcellulose, hydroxypropylmethyl cellulose, hydroxypropyl cellulose, gelatin, starch, pregelatinized starch and the like.

Suitable glidants of the present invention include calcium silicate, magnesium carbonate, magnesium oxide, magnesium silicate, talc, silicon dioxide, starch and the like.

Suitable lubricants of the present invention include sodium stearyl fumarate, magnesium stearate, calcium stearate, hydrogenated vegetable oil, stearic acid, glyceryl behenate, talc, and the like.

In another embodiment, the preferable extended release formulation according to present invention comprises about 40 to about 70% w/w of divalproex sodium, about 5 to about 20% w/w of one or more of hydrophilic polymer selected from the group consisting of hydroxypropylmethylcellulose (Methocel E15), hydroxyethylcellulose (Natrosol®), hydroxypropylcellulose (Klucel), polyvinylpyrrolidone (Kollidon), polyethylene oxide (Polyox®); about 2 to about 20% w/w of hydrophobic polymer selected from the group consisting of ethyl cellulose (Ethocel), polymethacrylic acid esters copolymer (Eudragit NE30D or Eudragit NE40D); about 15 to about 30% w/w of diluent selected from the group consisting of pregelatinised starch, microcrystalline cellulose, lactose; and about 0.5 to about 5% w/w of glidant selected from the group consisting of colloidal silicon dioxide and magnesium silicate.

In another embodiment of the present invention, the dosage form of the extended release formulations include tablets or capsules. The tablets may be uncoated or optionally coated. The film coating composition comprises a solution/suspension of film coating polymers and one or more excipients such as lactose, titanium dioxide, solubilizing agent and antisticking agent.

Suitable film coating polymers used according to the present invention are selected from ethylcellulose, hydroxypropylcellulose, hydroxypropylmethyl cellulose and the like or mixtures thereof.

The solubilizing agent of the present invention may be selected from anionic or non-ionic surfactants such as sodium lauryl sulphate, polyethylene-propylene glycol copolymer (poloxamer), polyoxyethylene stearates (Macrogol-stearate), poly sorbates, propylene glycol, and the like or mixtures thereof.

Suitable anti-sticking agents used according to the present invention are selected from talc, magnesium stearate and the like or a mixture thereof.

In another embodiment, the present invention also provides an extended release formulation comprising about 40 to about 70% w/w of divalproex sodium, about 5 to about 20% w/w of one or more of hydrophilic polymer selected from the group consisting of hydroxypropylmethylcellulose (Methocel E15), hydroxyethylcellulose (Natrosol®), hydroxypropylcellulose (Klucel), polyvinylpyrrolidone (Kollidon), polyethylene oxide (Polyox®); about 2 to about 20% w/w of hydrophobic polymer selected from the group consisting of ethyl cellulose (Ethocel), polymethacrylic acid esters copolymer (Eudragit NE30D or Eudragit NE40D); about 15 to about 30% w/w of diluent selected from the group consisting of pregelatinised starch, microcrystalline cellulose, lactose; and about 0.5 to about 5% w/w of glidant selected from the group consisting of colloidal silicon dioxide and magnesium silicate; which exhibits the following dissolution profile when measured in a type 2 dissolution apparatus (paddle) at 100 rpm, at a temperature of 37±0.5° C., in 500 ml of 0.1 N HCl for 45 minutes, followed by 900 ml of 0.05M phosphate buffer containing 75 mM sodium laurel sulfate (pH5.5):

i. not more than about 30% of total valproate is released after 3 hours of measurement;

ii. from about 30 to about 70% of total valproate is released after 9 hours of measurement;

iii. from about 50 to about 90% of total valproate is released after 12 hour of measurement, and;

iv. not less than 85% of total valproate is released after 18 hours of measurement.

In another embodiment, the extended release formulations of the present invention are prepared either by granulation technique or direct compression.

A preferable process for the preparation of an extended release formulation comprising divalproex sodium dispersed in a polymeric matrix comprising of about 5 to about 20% w/w of hydrophilic polymer, about 2 to about 20% w/w of hydrophobic polymer and about 15 to about 30% w/w of diluent comprises the following steps:

i) blending divalproex sodium with hydrophilic polymer, hydrophobic polymer and other excipients,

ii) granulating the blend of step (i),

iii) drying the granules obtained in step (ii),

iv) blending the dried granules of step (iii) with glidant,

v) compressing the blend of step (iv) into tablets or filled into capsules and

vi) finally coating the tablets of step (v) with film forming materials.

The solvents used for granulation according to the present invention are selected from water, isopropyl alcohol, ethanol, acetone, methylene chloride and the like or mixture thereof.

In yet another embodiment, the present invention also provides method of treating complex partial seizures, mania associated with bipolar disorders, and/or migraine headaches by administering extended release formulation prepared according to present invention.

The following examples further exemplify the invention and are not intended to limit the scope of the invention. It is obvious to those skilled in the art to find out the composition for other dosage forms and substitute the equivalent excipients as described in this specification or with the one known to the industry.

### EXAMPLE 1

### Divalproex Sodium Extended Release Tablets

| Ingredients | Qty/tab (mg) |
|---|---|
| Divalproex sodium | 546 |
| Hydroxypropylmethylcellulose | 140 |
| Ethyl cellulose | 135 |
| Pregelatinised starch | 174 |
| Ethyl cellulose | 15 |
| Colloidal silicon dioxide | 30 |
| Isopropyl alcohol | qs |
| Film coating | |
| Opadry | 30 |
| Isopropyl alcohol | qs |

The processing steps involved were given below:

i) divalproex sodium, hydroxypropylmethylcellulose, ethyl cellulose and pregelatinised starch were blended,

ii) binder solution of ethylcellulose in isopropyl alcohol was prepared,

iii) granulated the blended material of step (i) with binder solution of step (ii) and dried the granulated mass,

iv) granules of step (iii) were blended with colloidal silicon dioxide,

v) compressed the blend of step (iv) into tablets and

vi) tablets of step (v) was then coated with a solution/suspension of opadry in isopropyl alcohol.

The compositions disclosed in examples 2-4 were prepared by the similar procedure described in example 1.

### EXAMPLE 2

| Ingredients | Qty/tab (mg) |
|---|---|
| Divalproex sodium | 546 |
| Hydroxypropylmethylcellulose | 45 |
| Polyethylene oxide | 100 |
| Pregelatinised starch | 229 |
| Ethyl cellulose | 20 |
| Colloidal silicon dioxide | 30 |
| Isopropyl alcohol | qs |
| Film coating | |
| Opadry | 30 |
| Isopropyl alcohol | qs |

### EXAMPLE 3

| Ingredients | Qty/tab (mg) |
|---|---|
| Divalproex sodium | 538 |
| Hydroxypropylmethylcellulose | 45 |
| Polyethylene oxide | 100 |
| Pregelatinised starch | 259.5 |
| Ethyl cellulose | 7.5 |
| Silicon dioxide | 30 |
| Isopropyl alcohol | Qs |
| Film coating | |
| Opadry | 30 |
| Isopropyl alcohol | qs |

### EXAMPLE 4

| Ingredients | Qty/tab (mg) |
|---|---|
| Divalproex sodium | 538 |
| Hydroxypropylmethylcellulose | 145 |
| Pregelatinised starch | 269.5 |
| Ethyl cellulose | 7.5 |
| Colloidal silicon dioxide | 20 |
| Isopropyl alcohol | Qs |
| Film coating | |
| Opadry | 30 |
| Isopropyl alcohol | Qs |
| Purified water | Qs |

### EXAMPLE 5

| Ingredients | Qty/tab (mg) |
|---|---|
| Divalproex sodium | 538 |
| Hydroxypropylmethylcellulose | 145 |
| Eudragit NE 30 D | 157 |
| Pregelatinised starch | 250 |
| Colloidal silicon dioxide | 20 |
| Film coating | |
| Opadry | 30 |
| Isopropyl alcohol | Qs |
| Purified water | Qs |

The processing steps involved were as given below:

i) divalproex sodium, hydroxypropylmethylcellulose and pregelatinised starch were blended,

ii) granulated the blended material of step (i) with Eudragit NE 30D and dried the granulated mass,

iii) granules of step (ii) were blended with colloidal silicon dioxide,

iv) compressed the blend of step (iii) into tablets and

v) tablets of step (iv) was then coated with a solution/suspension of opadry in isopropyl alcohol and or purified water.

Table given below show the comparative dissolution profile of Divalproex sodium extended release tablets and DEPAKOTE ER® carried out in 500 ml of 0.1 N HCl for 45 minutes, followed by 900 ml of pH 5.5 phosphate buffer with 75 mM Sodium lauryl sulfate (SLS) according to the procedure described in the USP, Apparatus USP II Paddle, @ 100 rpm speed. The release profile (% of drug released in minutes) is given in table 1.

TABLE 1

| | DEPAKOTE ® ER | | |
|---|---|---|---|
| Time | (B. No. 40018AA22) | Example 4 | Example 5 |
| (Hrs) | % drug release | | |
| 3 | 21 | 20 | 20 |
| 9 | 47 | 51 | 49 |
| 12 | 58 | 64 | 60 |
| 18 | 99 | 104 | 77 |

We claim:

1. An extended release formulation comprising divalproex sodium dispersed in a polymeric matrix comprising of about 5 to about 20% w/w of hydrophilic polymer, about 2 to about 20% w/w of hydrophobic polymer and about 15 to about 30% w/w of diluents selected from calcium phosphate, calcium sulfate, microcrystalline cellulose, mannitol, pregelatinised starch or sorbitol, wherein the formulation is prepared by wet granulation.

2. The extended release formulation as claimed in claim 1, wherein the hydrophilic polymer is selected from polyvinylpyrrolidone, hydroxypropylcellulose, hydroxypropylmethylcellulose, methylcellulose, hydroxyethylcellulose, vinyl acetate copolymers, alginic acid and its salts, xanthan gum, polyethylene oxide or combination thereof.

3. The extended release formulation as claimed in claim 1, wherein the hydrophobic polymer is selected from ethyl cellulose, cellulose acetate, polymethacrylic acid esters copolymer, copolymers of polyvinyl alcohol and high molecular weight polyvinylalcohols, glyceryl behenate, glyceryl palmitostearate, glyceryl monostearate, glyceryl distearate or combination thereof.

4. (canceled)

5. The extended release formulation as claimed in claim 1, further comprises one or more excipients selected from binder, glidant and lubricant.

6. The extended release formulation as claimed in claim 5, wherein the binder is selected from polyvinylpyrrolidone, xanthan gum, carboxymethylcellulose, methylcellulose, ethyl cellulose, hydroxypropylmethylcellulose, hydroxypropyl cellulose, gelatin, starch and pregelatinized starch.

7. The extended release formulation as claimed in claim 5, wherein the glidant is selected from calcium silicate, magnesium carbonate, magnesium oxide, magnesium silicate, talc, silicon dioxide or starch.

8. The extended release formulation as claimed in claim 5, wherein the lubricant is selected from sodium stearyl fumarate, magnesium stearate, calcium stearate, hydrogenated vegetable oil, stearic acid, glyceryl behenate or talc.

9. An extended release formulation comprising:
i) about 40 to about 70% w/w of divalproex sodium;
ii) about 5 to about 20% w/w of one or more of hydrophilic polymer selected from the group consisting of hydroxypropylmethylcellulose, hydroxyethylcellulose, hydroxypropylcellulose, polyvinylpyrrolidone, polyethylene oxide,
iii) about 2 to about 20% w/w of hydrophobic polymer selected from the group consisting of ethyl cellulose, polymethacrylic acid esters copolymer,
iv) about 15 to about 30% w/w of diluent selected from the group consisting of pregelatinized starch, microcrystalline cellulose, lactose, and
v) about 0.5 to about 5% w/w of glidant selected from the group consisting of colloidal silicon dioxide and magnesium silicate,
wherein the formulation is prepared by wet granulation.

10. An extended release formulation comprising divalproex sodium dispersed in a polymeric matrix comprising of about 5 to about 20% w/w of hydrophilic polymer selected from polyvinylpyrrolidone, hydroxypropylcellulose, hydroxypropylmethylcellulose, methylcellulose, hydroxyethylcellulose, vinyl acetate copolymers, alginic acid and its salts, xanthan gum and polyethylene oxide, about 2 to about 20% w/w of hydrophobic polymer selected from ethyl cellulose, cellulose acetate, polymethacrylic acid esters copolymer, copolymers of polyvinyl alcohol and high molecular weight polyvinylalcohols, glyceryl behenate, glyceryl palmitostearate, glyceryl monostearate and glyceryl distearate, and about 15 to about 30% w/w of diluent selected from calcium phosphate, calcium sulfate, microcrystalline cellulose, mannitol, pregelatinised starch or sorbitol, wherein the formulation is prepared by wet granulation, which exhibits the following dissolution profile when measured in a type 2 dissolution apparatus (paddle) at 100 rpm, at a temperature of 37±0.5° C., in 500 ml of 0.1 N HCl for 45 minutes, followed by 900 ml of 0.05 M phosphate buffer containing 75 mM sodium laurel sulfate at a pH 5.5:
i) not more than 30% of total valproate is released after 3 hours of measurement,
ii) from about 30 to 70% of total valproate is released after 9 hours of measurement,
iii) from about 50 to about 90% of total valproate is released after 12 hour of measurement, and
iv) not less than 85% of total valproate is released after 18 hours of measurement.

11. (canceled)

12. A method of treating complex partial seizures, mania associated with bipolar disorders, and/or migraine headaches by administering extended release formulation of claim 1.
